(19) United States
(12) Patent Application Publication
(10) Pub. No.: US 2025/0082653 A1
(43) Pub. Date: Mar. 13, 2025

(54) THERAPEUTIC PRODUCT IN THE FORM OF A DENTAL GEL

(71) Applicant: CB21 PHARMA, S.R.O., Brno (CZ)

(72) Inventors: Jan STORCH, Praha 10 (CZ); Jan VACEK, Olomouc (CZ); Lenka PRUSOVA, Kladno (CZ)

(21) Appl. No.: 18/580,273

(22) PCT Filed: Jun. 13, 2022
(86) PCT No.: PCT/CZ2022/050057

§ 371 Date: Jan. 18, 2024

(30) Foreign Application Priority Data
Aug. 7, 2021 (CZ) PV 2021-369

(51) Int. Cl. A61K 31/00; A61K 9/00; A61K 9/06; A61K 47/06; A61K 47/10; A61K 47/14; A61K 47/18; A61K 47/36; A61P 1/02

(52) U.S. Cl. CPC A61K 31/658; A61K 9/0063; A61K 9/06; A61K 47/06; A61K 47/10; A61K 47/14; A61K 47/18; A61K 47/36; A61P 1/02

(57) ABSTRACT

The invention provides a therapeutic product for dental use, which comprises an active ingredient cannabidiol and it further comprises squalane as solvent, wherein the product is in the form of dental gel. The product may contain hyaluronic acid or a pharmaceutically acceptable salt thereof. The product is suitable in particular for treatment and/or prevention of gingival inflammation, periodontitis and periimplantitis.

### FIELD OF ART

The present invention relates to a therapeutic product in the form of a dental gel, suitable in particular for adjuvant therapy of healing and inflammation of the gingiva.

### BACKGROUND ART

Supra/subgingival plaque formation and its calcification leads to the formation of supra/sub-gingival calculus. Accumulation of the calculus disrupts the dentogingival complex/junction, which is the cause of non-bacterial gingival inflammation (gingivitis). In an individual with an impaired immune system, innate genetic predisposition, mental health problems, or poorly performed oral hygiene, oral ecology is disturbed and changes in the composition of the oral microflora occur, including colonization of the gingiva with periodontopathogenic anaerobic bacteria. Periopathogens include Aggregatibacter actinomycetemcomitans, Filifactor alocis, Fusobacterium nucleatum, Porphyromonas gingivalis, Prevotela intermedia, Tannerella forsythia, Treponema denticola, Selenomonas artemidis, and Veillonella dispar, as well as several species of streptococci, most notably Streptococcus oralis, S. reptococcus, S. mitis, S. sanguis, and S. mutans. Virulent and proinflammatory factors of these bacteria activate monocytes, macrophages and neutrophils/leukocytes, key cells of the oral immune system, to massively produce proinflammatory cytokines and chemokines (Pandit N, Changela R, Bali D, Tikoo P, Gugnani S. Porphyromonas gingivalis: Its virulence and vaccine. J Int Clin Dent Res Organ 2015; 7:51-8). The inflammatory process caused by the periopathogens destroy these bacteria and destructs the periodontal tissue, especially periodontal ligament fibroblasts, odontoblasts and alveolar bone osteoblasts. This stage of inflammation and gingival disease is classified as acute or chronic periodontitis (Kinane D F, Mobelli A. Periodontal disease. Frontiers of oral biology Vol. 15. S. Karger A G, Basel 2012, ISBM 978-3-8055-9833-0).

Gingivitis or periodontitis, respectively, is a health problem that affects 20 to 50% of the world's population (Nazir M A. Prevalence of periodontal disease, its association with systemic diseases and prevention. Int J Health Sciences 2016; 1:1-9). Oral hygiene products (toothpastes, gels and mouthrinses) should contain synthetic or natural substances with effects on inhibiting bacterial biofilm formation and subsequent plaque calcification, provide an anti-inflammatory and healing effect on the gingiva, bacteriostatic or bactericidal effect on pathogenic bacteria with no or minimal effect on commensal bacteria of the oral microflora. The use of such products in daily oral hygiene can significantly reduce the risk or course of periodontal disease (Pihstrom B L, Michalowicz B S, Johnson N W. Periodontal diseases. Lancet 2005; 366:1809-1820).

Inorganic salts such as sodium fluoride, zinc chloride/sulphate, tin fluoride or aluminum lactate are active components in cosmetic/therapeutic products for the chemoprophylaxis of the oral cavity. Synthetic organic compounds useful as such active components include amine fluoride, cetylpyridinium chloride, octenidine, hexetidine and a problematic substance triclosan. The most common chemoprophylactic in oral hygiene products used by both patients and dentists is chlorhexidine and its salts chlorhexidine gluconate or digluconate (Shapiro S, Guggenheim B, Chemoprophylaxis in the oral cavity: “plus on change les choses, plus elles devraient rester les memes” in Oral Biology at the Turn of the Century (Edits. Guggenheim B, Shapiro S) S. Karger A G. pp. 226-238. Zurich 1998). Chlorhexidine is effective against a wide range of Gram-positive and Gram-negative bacteria, yeasts, some fungi and viruses. Its application in the form of a gel (1%) may be accompanied by a number of side effects, such as increased calculus formation, hyperkeratosis of the taste buds of the tongue, desquamation of the epidermis of the oral mucosa and discolouration of the tooth enamel (James P, Worthington H V, Parnell C, Harding M, Lamont T, Cheung A, Whelton H, Riley P. Chlorhexidine mouthrinse as an adjunctive treatment for gingival health. Cochrane Database of Systematic Reviews 2017, Issue 3. Art. No.: CD008676. DOI: 10.1002/14651858.CD008676.pub2.).

Complex medicinal plant extracts or individual phytoceutics (with anti-inflammatory effects, antimicrobial effects against Gram-positive and/or Gram-negative bacteria and anti-adherent effects on the formation of bacterial biofilm on the mucosa and solid structures in the oral cavity) are common components in oral hygiene products as well (Groppo F C, Bregamaschi CdeCC, Cogo K, Franz-Montan M, Motta R H, de Andrade ED. Use of phytotherapy in dentistry. Phytother Res. 2008; 22:993-998.; Petti S, Scully C. Polyphenols, oral health and diseases: A review. J Dentistry 2009; 37“413-423; Hirasawa M, Takada K, Otake S. Inhibition of acid production in dental plaque bakteria by the green tea catechins. Carries Res. 2006; 40:265-270; Feghali K, Feldman M, Vu Dang La, Santos J, Grenier D. Cranberry proanthocyanidins: Natural Weapons against periodontal diseases. J Agric Food Chem 2012; 60:5728-35.).

A phytoceutical for which there is still little clinical data on its effect on the gingiva and periodontium is cannabidiol (CBD), a non-psychotropic major component of a complex plant extract of the genus Cannabis. CBD is an agonist of the CB2 receptor of the endocannabinoid system which, upon activation, inhibits the production of proinflammatory cyto- and chemokines, macrophages and neutrophils/leukocytes of the mucosal immune system (Gu Z, Singh S, Niyogi R G, Lamont G J, Wang H, Lamont R J, Scott D A. Marijuana-Derived Cannabinoids Trigger a CB2/PI3K Axis of Suppression of the Innate Response to Oral Pathogens, Front Immunol 2019; 10: Article 2288). The anti-inflammatory action of CBD on soft tissues via modulating the immune response may be beneficial for patients after dental surgery or in patients with periodontal problems or for reducing alveolar bone tissue resorption. Recently, CBD has been shown to reduce the secretion of bacterial outer membrane vesicles, while inhibiting communication between the bacteria and thus biofilm formation with commensal bacteria of the host (Kosgodage U S, Matewele P, Awamaria B, Kraev I, Warde P, Mastroianni G, Nunn A V, Guy G W, Bell J D, Inal J M, Lange S. Cannabidiol Is a Novel Modulator of Bacterial Membrane Vesicles. Front Cell Infect Microbiol 2019; 9: Article 324). On the other hand, it has recently been observed that the use of cannabis and cannabis preparations that contain CBD, or its combination with Δ9-THC, causes health problems in periodontal soft tissues (a. Rawal S Y, Tatakis D N, Tipton D A. Periodontal and oral manifestations of marijuana use. J Tenn Dent Assoc 2012; 92:26-31, b. Park J B, Jung K M, Piomelli D, J. Periodontal Implant. Sci. 2020, 50, 355). CBD can also be toxic to commensal bacteria in the oral cavity, disrupting oral ecology and causing changes in the composition of the oral microflora and gingival population by periodontopathogenic anaerobic bacteria and facultatively anaerobic bacteria. The anti-inflammatory effect of CBD further attenuates the immune response against these pathogens (P. Jirasek, A. Jusku, V. Simanek, J. Frankova, J.

Storch, J. Vacek, Cannabidiol and periodontal inflammatory disease: A critical assessment, Biomed. Pap. 166 (2022) 155-160). As a result, there is an uncontrolled development of the inflammatory process, or the progression of an already existing one.

### DISCLOSURE OF THE INVENTION

The present invention relates to a therapeutic product for dental use which comprises an active ingredient consisting of cannabidiol (CBD) and the solvent squalane. Squalane has a key role in the product, which is reflected in the effective permeation of CBD through the mucosa/skin, thus enabling its absorption into the gingival fibroblasts. This mechanism reduces the CBD concentration on the gingival surface and results in a significant reduction of the negative effects of cannabidiol on the composition of the oral microflora and oral microenvironment. The therapeutic product is in the form of a dental gel.

In a preferred embodiment, the preparation also includes hyaluronic acid or a pharmaceutically acceptable salt thereof. The hyaluronic acid reduces the cytotoxicity of cannabidiol. This allows to increase the CBD concentration in the product by up to two orders of magnitude, compared to its IC₅₀ values for human gingival fibroblasts.

The therapeutic product according to the present invention is suitable for users/patients with chronic gingival inflammation or periodontitis caused by immune system disruption, innate disposition and/or poor lifestyle (diet, alcohol, smoking, stress) and periimplantitis. The beneficial pharmacological effect of the gel is a combination of the CBD and squalane excipient actions, and preferably also the action of hyaluronic acid or its pharmaceutically acceptable salt, and is manifested by reduction in plaque formation and gingival bleeding, by periodontal pocket healing and by decreasing the amount of periodontopathogenic bacteria. In the soft tissues of the oral cavity, the active ingredient has a regulatory function in the inflammatory response induced mechanically or by periodontopathogenic bacteria.

The product does not show any undesirable side effects and is suitable for long-term (oral hygiene) chemoprophylactic therapy in addition to professional periodontal treatment. Due to a low or zero content of chlorhexidine or its derivatives, for preservation purposes only, the product does not show any side effects associated with this substance. The complex effect of one or both active components in combination with squalane results in a significant improvement in the course of gingival and periodontal inflammation or delay of the reinfection.

Preferably, cannabidiol and hyaluronic acid or a pharmaceutically acceptable salt thereof are present in the weight ratio within 20:1 to 1:1, more preferably 20:1 to 5:1, even more preferably 15:1 to 8:1.

The therapeutic product according to the invention may preferably contain 0.1 to 5 weight % of cannabidiol, more preferably 0.5 to 2 weight % of cannabidiol, and optionally up to 0.3 weight % chlorhexidine gluconate or digluconate.

The therapeutic product according to the invention may preferably contain 0.1 to 5 weight % of cannabidiol, 0.01 to 2 weight % of hyaluronic acid or a pharmaceutically acceptable thereof, and optionally up to 0.3 weight % chlorhexidine gluconate or digluconate.

In a preferred embodiment, the therapeutic product according to the invention may contain 0.5 to 2 weight % of cannabidiol, 0.05 to 1 weight % of hyaluronic acid or a pharmaceutically acceptable thereof.

Hyaluronic acid or a pharmaceutically acceptable thereof preferably has a low to medium molecular weight within the range of 200 to 1800 kDa.

The pharmaceutically acceptable salt of hyaluronic acid is typically a sodium salt or a potassium salt. Sodium salt of hyaluronic acid is preferred for use in the dental gel.

The dental gel according to the present invention further comprises at least one auxiliary substance. Auxiliary substances include, in particular, gelling agents, solvents, water, stabilizers and flavorings.

The gelling agent may be, for example, glycerin, polyethylene glycol, acrylic acid-based polymers (carbomers), and mixtures thereof.

The solvent for dissolving cannabidiol is squalane. Squalane is a hydrating lipophilic substance which further improves the healing and calming of the gums. Simultaneously, squalane is a suitable solvent for cannabidiol and is suitable for achieving its homogenization with the remaining components of the gel. Squalane further decreases the cannabidiol concentration on the surface of the gums. Weight ratio of squalane to cannabidiol is preferably within the range of 2:1 to 20:1, more preferably up to 10:1 or 3:1 to 5:1.

The therapeutic product according to the invention may preferably contain 0.1 to 5 weight % of cannabidiol, 0.01 to 2 weight % of hyaluronic acid or a pharmaceutically acceptable salt thereof, 1 to 10 weight % of squalane, 5 to 40 weight % of gelling substances, and water q.s. 100 weight %.

The product may further contain up to 0.3 weight % of chlorhexidine gluconate or digluconate, preferably up to 0.1 weight % of chlorhexidine gluconate or digluconate.

More preferably, the therapeutic product may contain 0.5 to 2 weight % of cannabidiol, 0.05 to 1 weight % of hyaluronic acid or a pharmaceutically acceptable salt thereof, 3 to 8 weight % of squalane, 5 to 20 weight % of gelling agent(s), and water q.s. to 100 weight %.

### BRIEF DESCRIPTION OF DRAWINGS

FIG. 1. Cytotoxicity of CBD expressed as a percentage of control determined by MTT method in human gingival fibroblasts (n=6)—Example 2.

FIG. 2. IL-6 production after 6/24-hour application of CBD on human gingival fibroblasts (C=control, without CBD)—Example 2.

FIG. 3. IL-8 production after 6/24-hour application of CBD on human gingival fibroblasts (C=control, without CBD)—Example 2.

FIG. 4. Production of MMP-2 after 6/24-hour application of CBD on human gingival fibroblasts (C=control, without CBD)—Example 2.

FIG. 5. Increase in CBD concentration observed in human gingival fibroblast lysates after their 6 h and 24 h incubation with 0.5 μM CBD. Determined by UHPLC/MS method. CBD was not detected in the culture medium—Example 5.

FIG. 6. Permeation of CBD from dental gels containing various lipophilic components (columns 2 and 3) and without any lipophilic component (column 1) through Strat-M® synthetic human skin after 24 h. Determined by UHPLC/MS-Example 6.

### EXAMPLES OF CARRYING OUT THE INVENTION

### Example 1. Various Compositions and Preparation of Dental Gel

| | E1a | E1b |
|---|---|---|
| Component | weight % | weight % |
| Carbopol Ultrez 10 NF polymer | 0.50 | 0.50 |
| Glycerine | 10.00 | 10.00 |
| Squalane | 4.00 | 4.00 |
| Cannabidiol | 1.00 | 1.00 |
| Sodium hyaluronate | 0.10 | — |
| Chlorhexidine digluconate | 0.02 | 0.02 |
| Purified water | 84.38 | 84.48 |

Procedure for preparation of E1a:

1. Sodium hyaluronate is dissolved in ca. 10% of the total amount of water to form a transparent solution.

2. Carbopol Ultrez 10 NF polymer is hydrated in the appropriate amount of water to form a whitish suspension.

3. Glycerine, chlorhexidine digluconate and the solution from step 1. are added to the suspension from step 2. The mixture is stirred.

4. pH of the mixture obtained in step 3. is adjusted by triethylamine to the value pH=6.0. Transparent gel is formed.

5. CBD is dissolved in squalane under heating to max. 60° C.

6. The squalane solution of CBD is mixed with the gel obtained in step 4., and the mixture is stirred.

7. To achieve a perfect dispersion of CBD in the gel, the mixture is homogenized at min. 3000 RPM for approx. 15 seconds.

Procedure for preparation of E1b:

1. Carbopol Ultrez 10 NF polymer is hydrated in the appropriate amount of water to form a whitish suspension.

2. Glycerine and chlorhexidine digluconate are added to the suspension. The mixture is stirred.

3. pH of the mixture obtained in step 2. is adjusted by triethylamine to the value pH=6.0. Transparent gel is formed.

4. CBD is dissolved in squalane under heating to max. 60° C.

5. The squalane solution of CBD is mixed with the gel obtained in step 3., and the mixture is stirred.

6. To achieve a perfect dispersion of CBD in the gel, the mixture is homogenized at min. 3000 RPM for approx. 15 seconds.

### Example 2. Effects of Cannabidiol on Human Gingival Fibroblasts

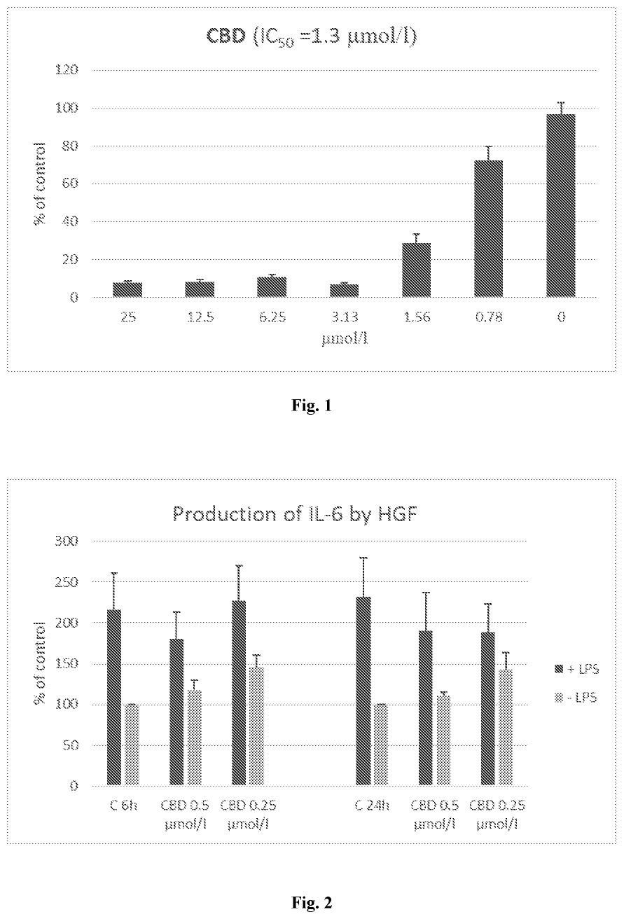

In an in vitro study of the effect of cannabidiol (CBD), human gingival fibroblasts (HGF) isolated from gingiva taken from volunteers at the Department of Oral, Maxillofacial and Facial Surgery of the University Hospital and Medical Faculty of Palacký University Olomouc approved by the Ethics Committee of the University Hospital and Medical Faculty of Palacký University Olomouc (2019) were used. In the first phase of the experiment, a safe CBD concentration was determined based on the results of MTT, which is cytotoxicity test based on the reduction of 3-(4,5-dimethylthiazol-2-yl)-2,5-diphenyltetrazolium bromide (MTT) to the corresponding formazane. The test was performed according to M. D. Maines, L. G. Costa, D. J. Reed, et al. Current protocols in toxicology. New York: John Wiley & Sons. (1998). The test results are shown in FIG. 1.

Cells seeded in a 96-well plate at a count of 3×10⁴ cells/ml and 100 μl/well, which were allowed to adhere for 24 h and then incubated with CBD for 24 h in a concentration range of 25 to 0.78 μmol/1, 100 μl MTT was added (0.5 mg/ml in serum-free culture medium), the mixture was incubated for 2 h. After the incubation period and aspiration of the MTT mixture, 100 μl DMSO (dimethyl sulfoxide) with NH₃ (1%, v/v) was added. Absorbance was measured at 540 nm.

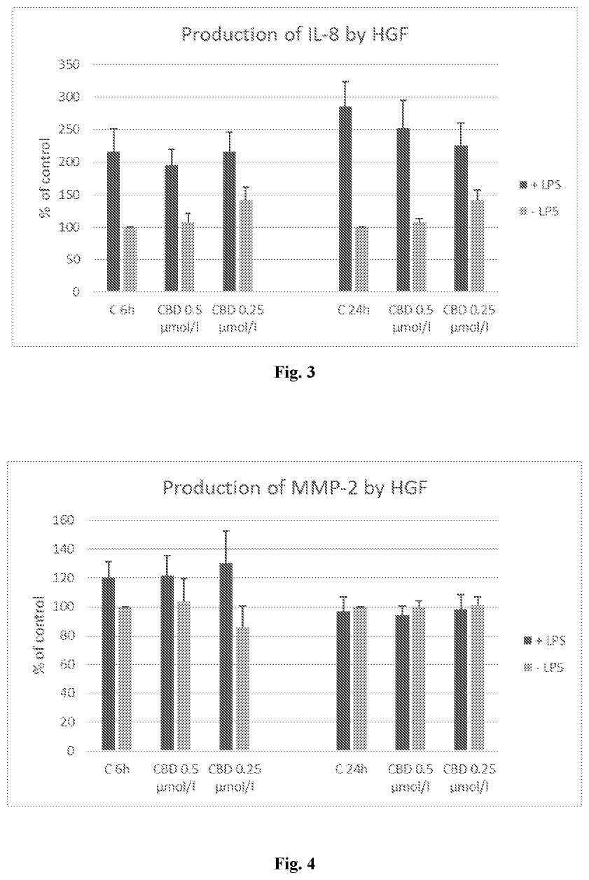

According to the values obtained from the MTT test, two subtoxic concentrations of CBD (0.25 and 0.5 μmol/l) were selected and used to determine its anti-inflammatory effect. Inflammation to HGF was induced by P. gingivalis lipopolysaccharide (LPS) according to How K. Y., Song K. P., Chan K. G., Porphyromonas gingivalis: An Overview of Periodontopathic Pathogen below the Gum Line. Front Microbiol 2016; 7:53. HGFs were seeded in 6-well plates and allowed to grow to confluence. The cells were washed with 1 ml of PBS, and then LPS (1 μg/ml, 2 ml/well) in serum-free culture medium was added, and incubated for 24 hours. A portion of the cells was left without the application of LPS (i.e., without induced inflammation). At the end of the incubation period, CBD in serum-free culture medium at concentrations of 0.25 and 0.5 μmol/l was added (2 ml/well) for 6 and 24 hours, both for cells cultured with LPS and for cells without LPS application. At selected time intervals, the culture medium was collected and stored at −80° C. for further analysis. The levels of proinflammatory interleukins IL-6, IL-8 (Human ELISA Development Kit, Cat. No. 900-K16 and 900-K18, PeproTech, Baria, Czech Republic) and matrix metalloproteinase (MMP-2) (Duo Set ELISA Development System, Cat. No. DY902, R&D, Bio-Techne, UK) were determined by ELISA. The selected parameters play a major role in pathological processes and play an important role in periodontal diseases (Noh K. M., Jung M., Kim S. H., Lee S. R., Park K. H., Kim D. H., Kim H. H., Park Y. G. Assessment of IL-6, IL-8 and TNF-α levels in the gingival tissue of patients with periodontitis. EXP Ther Med 2013; 6(3):847-851). The cytokine IL-6 plays a role in the inflammatory response. In persistent inflammation, the level of MMP-2 increases, which significantly contributes to tissue destruction. The inflammatory mediator IL-8 also acts as a chemoattractant. In an experiment after LPS inflammation induction and subsequent administration of cannabidiol, decrease in the levels of IL-6 (FIG. 2) and IL-8 (FIG. 3) was observed. There were no significant changes in the MMP-2 levels (FIG. 4). In conclusion, our experiments confirmed the anti-inflammatory effect of both doses of cannabidiol, especially in the longer time perspective of 24-h incubation.

### Example 3. Testing of Antimicrobial Effects of Cannabidiol

Strains Streptococcus mutans CCM 7409 and Porphyromonas gingivalis CCM 3985 were selected for testing. Both were obtained from Czech Collection of Microorganisms (Brno). BHI+kh medium (Brain-heart infusion (Oxoid, UK) enriched with vitamin K (0.001 mg/ml) and hemin (5 mg/ml)) was used to cultivate S. mutans, and for P. gingivalis medium WCHA—Wilkins-Chalgren agar (HiMedia, India), with 7% sheep erythrocytes and vitamin K (0.001 mg/ml). (Blaskovich M A T, Kavanagh A M, Elliott A G, Zhang B, Ramu S, Amado M et al. The antimicrobial potential of cannabidiol. Commun Biol 2021; 4(1):1-18).

The susceptibility of selected microbes to tested substance was verified by the microdilution method according to Clinical and Laboratory Standards Institute (M Methods for Antimicrobial Susceptibility Testing of Anaerobic Bacteria. 9^{th} ed. CLSI standard M11. Wayne, PA: Clinical and Laboratory Standards Institute; 2018).

Basic working solutions were prepared from the tested substance by dilution in DMSO. These working solutions were then further diluted with BHI+kh medium (Brain-heart infusion (Oxoid, UK) enriched with vitamin K (0.001 mg/ml) and hemin (5 mg/ml)) so that DMSO content was at least 1:100 (w/w) and thus did not inhibit microbial growth. The required concentrations of the tested substance were then prepared by serial dilution, dilution factor 1:2. 135 μl of each dilution was pipetted into the wells of a 96-well round-bottomed hardened polystyrene microtiter plate (Gamma České Budějovice, a.s.).

From fresh cultures of the test strains on Wilkins-Chalgren agar (WCHA; HiMedia, India), with 7% sheep erythrocytes and vitamin K, suspensions were prepared in BHI+kh at an optical density corresponding to 0.5 degrees McFarland turbidity scale (after subtraction of optical density of the medium). The suspensions were then diluted in the ratio 1:15 (final inoculum concentration 5×10⁶ CFU/ml). Individual wells with 135 μl of the CBD solutions and control wells were inoculated with 15 μl of the prepared inoculum.

The inoculated microtiter plates were incubated in an Anaerobic Work Station (Ruskinn Technology, UK) with an anaerobic atmosphere (80% N₂, 10% CO₂ and 10% H₂) at 37° C. The incubation was performed for 60 hours for P. gingivalis and 24 hours for S. mutans.

The sterility of the medium was checked by transferring 150 μl BHI+kh to three wells of a microtiter plate. Three wells on each plate containing only 135 μL BHI+kh and 15 μl of the prepared inoculum served as a growth control for the test microbe.

After evaluating the MIC, the contents of the wells with the highest concentration of CBD and with visible microbial growth were inoculated onto WCHA. The inoculum itself was inoculated in the same way. After 48 hours (S. mutans) or 14 days (P. gingivalis) of incubation in an anaerobic atmosphere, the purity of the grown microbial culture was evaluated. In case of contamination, the result was considered invalid.

The growth of the microbe, which manifested itself by turbidity of the medium or as a sediment at the bottom of the well of the microtiter plate, was monitored in the individual wells of the microtiter plate. When inhibiting microbial growth, the medium remained clear, with no visible sedimentation or turbidity. The minimum inhibitory concentration (MIC) was determined as the lowest concentration of the substance at which no microbial growth was observed. The MIC of cannabidiol was performed on both independent microbes in three independent experiments, each with three replicates.

The results are summarized in Table 1.

TABLE 1
Minimum inhibition concentration values for
CBD for P. gingivalis and S. mutans.

| | |
|---|---|
| P. gingivalis | S. mutans |

| | MIC median | MIC range | MIC median | MIC range |
|---|---|---|---|---|
| | [μg/ml] | [μg/ml] | [μg/ml] | [μg/ml] |
| CBD | 1.5 | 1-2 | 16 | 16 |

### Example 4. Results from a Randomized, Double-Blind, Placebo-Controlled Clinical Trial “Pilot Study of the Effect of Cannabidiol on Chronic Periodontitis” Approved by the Ethics Committee of the University Hospital and the Medical Faculty of Palacký University (2020)

The study was performed with samples of dental gels of the following compositions:

Dental gel containing the active component CBD and squalane:

| Component | weight % |
|---|---|
| Carbopol Ultrez 10 NF polymer | 0.50 |
| Glycerine | 10.00 |
| Squalane | 4.00 |
| Cannabidiol | 1.00 |
| Sodium methylparaben | 0.15 |
| Purified water | 84.35 |

### Preparation Procedure:

1. Carbopol Ultrez 10 NF polymer is hydrated in water to form a whitish suspension.

2. Glycerine and sodium methylparaben are added to the suspension from step 1. The mixture is stirred.

3. pH of the mixture obtained in step 2. is adjusted by triethylamine to the value pH=6.0. Transparent gel is formed.

4. CBD is dissolved in squalane under heating to max. 60° C.

5. The squalane solution of CBD is mixed with the gel obtained in step 3., and the mixture is stirred.

6. To achieve a perfect dispersion of CBD in the gel, the mixture is homogenized at min. 3000 RPM for approx. 15 seconds.

### Placebo Mixture

| Component | weight % |
|---|---|
| Carbopol Ultrez 10 NF polymer | 0.50 |
| Glycerine | 10.00 |
| Squalane | 4.00 |
| Sodium methylparaben | 0.15 |
| Purified water | 85.35 |

### Preparation Procedure:

1. Carbopol Ultrez 10 NF polymer is hydrated in water to form a whitish suspension.

2. Glycerine and sodium methylparaben are added to the suspension from step 1. The mixture is stirred.

3. pH of the mixture obtained in step 2. is adjusted by triethylamine to the value pH=6.0. Transparent gel is formed.

4. Squalane is mixed with the gel obtained in step 3., and the mixture is stirred.

Recruitment and study design: Recruitment (men and women, age 35-65) was followed by several visits:

1. At the first visit (0 day), patients who meet the inclusion criteria will be documented via Russel's periodontal index and plaque index (Silness and Loe), microbiological sampling, removal of supra/sub-gingival plaque and calculus will be done. Furthermore, a gingival sample will be collected for histopathological examination and, hygienic instructions will take place.

2. At the second visit (day 7), the patients will be checked for proper oral hygiene, patient's periodontal health will be assessed via the set of periodontal, gingival and hygienic indices and CBD dental gel (or identical placebo without CBD) will be applied (5 min exposure). Toothpastes containing CBD or placebo will be handed over. The patients will use the toothpastes to substitute their normal dental hygiene for the duration of the study.

3. At the third visit (day 22), patient's periodontal health will be assessed via the set of periodontal, gingival and hygienic indices and CBD dental gel (or identical placebo without CBD) will be applied (5 min exposure).

4. At the fourth visit (day 37), in case of termination of therapy, gingival samples will be collected for histopathological examination. Patient's periodontal health will be assessed via the set of periodontal, gingival and hygienic indices. A microbiological sample will be collected as well. If the therapy is continued, hygienic re-instruction and repeated application of CBD gel will take place (5 min exposure).

5. During the last visit, patient's periodontal health will be assessed via the set of periodontal, gingival and hygienic indices. Microbiological sampling will be performed. The total duration of the study was set at 67 days.

Inclusion criteria: chronic periodontitis, age 35-65 years, number of native teeth 16 or 16+, signed informed consent, without physical or mental impairment.

Exclusion criteria: chronic diseases (diabetes mellitus, oncological diseases), increased bleeding (medications—anticoagulants, antiplatelet agents, bleeding diathesis), pregnant and lactating women, tabacco smokers, users of cannabis or cannabis products, ATB treatment during the last 3 months, parallel participation in another clinical trial, patient with removable prosthesis.

Evaluation of study results: The clinical study will be evaluated based on Russell's periodontal index, plaque index (Silness and Loe), gingival index (Loe and Silness), gingival bleeding index (Ainamo and Bay), modified gingival index (Lobene et al.) (John C. Greene, Jack R. Vermillion, The oral hygiene index: a method for classifying oral hygiene status, The Journal of the American Dental Association, 61, 2, 1960, 172-179). Statistical analysis will be performed in SPSS or STATA software. Furthermore, microbiological sampling and gingival samples for histopathological examination will be collected. A microbiological sample from the periodontal pocket was performed for examination according to the VariOr-Dento methodology.

The results showed that the use of the CBD+squalane product resulted in a significant improvement in periodontal indixes (example: patient B) compared to the patient with placebo (example: patient A), see Table 2. Patient B also had a reduction in the number of pathogenic bacteria in the oral cavity; see Tables 3 and 4.

As mentioned above, it has recently been observed that the use of CBD containing preparations cause health problems in periodontal soft tissues (a. Rawal S Y, Tatakis D N, Tipton D A. Periodontal and oral manifestations of marijuana use. J Tenn Dent Assoc 2012; 92:26-31, b. Park J B, Jung K M, Piomelli D, J. Periodontal Implant. Sci. 2020, 50, 355). CBD is toxic to commensal bacteria in the oral cavity, disrupting oral ecology and causing changes in the composition of the oral microflora and gingival population by periodontopathogenic anaerobic bacteria and facultatively anaerobic bacteria. The anti-inflammatory effect of CBD further attenuates the immune response against these pathogens (P. Jirasek, A. Jusku, V. Simanek, J. Frankova, J. Storch, J. Vacek, Cannabidiol and periodontal inflammatory disease: A critical assessment, Biomed. Pap. 166 (2022) 155-160). As a result, there is an uncontrolled development of the inflammatory process, or the progression of an already existing one. Therefore, it is essential that CBD does not remain on the surface of the gums but penetrates the tissue. The role of squalane was further supported in Example 6, describing diffusion of CBD through a synthetic skin model when dissolved in various solvents and also in Example 5 showing quick absorption by gingival fibroblasts. The clinical test according to Example 4 demonstrates the positive effect of the preparation. It is worth mentioning that without the presence of squalene this result would not have been achieved.

TABLE 2
Values of periodontal indices in patent A using placebo
and in patient B using the product containing CBD.

| | first | fifth | |
|---|---|---|---|
| | visit | visit | evaluation |
| Patient A | | | |
| Plaque index | 1.24 | 1.04 | no change |
| Modified gingival index | 0.68 | 0.92 | no change |
| Gingival bleeding index | 0.52 | 0.48 | no change |
| Gingival index | 1.32 | 1.40 | no change |
| Periodontal index | 2.64 | 2.96 | no change |
| Patient B | | | |
| Plaque index | 0.72 | 0.83 | no change |
| Modified gingival index | 1.16 | 0.62 | change from medium |
| | | | inflammation to |
| | | | mild inflammation |
| Gingival bleeding index | 0.60 | 0.33 | 50% decrease in bleeding |
| Gingival index | 1.48 | 1.04 | change from medium |
| | | | inflammation to |
| | | | mild inflammation |
| Periodontal index | 5.92 | 5.83 | no change |

TABLE 3
Example of periodontal pocket colonization in patient A using
the placebo at the 1^{st} (start of study) and 5^{th} (end of study)
visits. No apparent improvement in microbial colonization.

| | | | |
|---|---|---|---|
| Complex | Pathogens | Abbreviation | Finding |

First visit:

| | | | |
|---|---|---|---|
| Aa | Aggregatibacter actinomycetemcomitans | Aa | −− |
| Red | Porphyromonas gingivalis | Pg | +++ |
| | Tannerella forsythia (Bacteroides forsythus) | Tf | +++ |
| | Treponema denticola | Td | +++ |
| Orange | Parvimonas micra | Pm | +++ |
| | Prevotella intermedia | Pi | −− |
| | Fusobacterium nucleatum | Fn | +++ |
| Orange- | Campylobacter rectus | Cr | + |
| associated | Eubacterium nodatum | En | +++ |
| Green | Eikenella corrodens | Ec | +++ |
| | Capnocytophaga sp. | Cs | −− |

| | |
|---|---|
| Resistance to β-lactam antibiotics | − |

| | |
|---|---|
| Overall risk of resorption | High |

Fifth visit:

| | | | |
|---|---|---|---|
| Aa | Aggregatibacter actinomycetemcomitans | Aa | −− |
| Red | Porphyromonas gingivalis | Pg | +++ |
| | Tannerella forsythia (Bacteroides forsythus) | Tf | +++ |
| | Treponema denticola | Td | ++ |
| Orange | Parvimonas micra | Pm | +++ |
| | Prevotella intermedia | Pi | −− |
| | Fusobacterium nucleatum | Fn | +++ |
| Orange | Campylobacter rectus | Cr | + |
| associated | Eubacterium nodatum | En | ++ |
| Green | Eikenella corrodens | Ec | + |
| | Capnocytophaga sp. | Cs | + |

| | |
|---|---|
| Resistance to β-lactam antibiotics | − |

| | |
|---|---|
| Overall risk of resorption | High |

TABLE 4
Example of periodontal pocket colonization in patient B using the gel
containing CBD at the 1^{st} (start of study) and 5^{th} (end of study)
visits. Significant reduction of the number of pathogenic bacteria.

| | | | |
|---|---|---|---|
| Complex | Pathogens | Abbreviation | Finding |

First visit:

| | | | |
|---|---|---|---|
| Aa | Aggregatibacter actinomycetemcomitans | Aa | −− |
| Red | Porphyromonas gingivalis | Pg | +++ |
| | Tannerella forsythia (Bacteroides forsythus) | Tf | ++ |
| | Treponema denticola | Td | + |
| Orange | Parvimonas micra | Pm | +++ |
| | Prevotella intermedia | Pi | −− |
| | Fusobacterium nucleatum | Fn | +++ |
| Orange | Campylobacter rectus | Cr | −− |
| associated | Eubacterium nodatum | En | ++ |
| Green | Eikenella corrodens | Ec | ++ |
| | Capnocytophaga sp. | Cs | +++ |

| | |
|---|---|
| Resistance to β-lactam antibiotics | − |

| | |
|---|---|
| Overall risk of resorption | High |

Fifth visit:

| | | | |
|---|---|---|---|
| Aa | Aggregatibacter actinomycetemcomitans | Aa | −− |
| Red | Porphyromonas gingivalis | Pg | −− |
| | Tannerella forsythia (Bacteroides forsythus) | Tf | −− |
| | Treponema denticola | Td | −− |
| Orange | Parvimonas micra | Pm | +++ |
| | Prevotella intermedia | Pi | −− |
| | Fusobacterium nucleatum | Fn | +++ |
| Orange | Campylobacter rectus | Cr | ++ |
| associated | Eubacterium nodatum | En | ++ |
| Green | Eikenella corrodens | Ec | ++ |
| | Capnocytophaga sp. | Cs | ++ |

| | |
|---|---|
| Resistance to β-lactam antibiotics | − |

| | |
|---|---|
| Overall risk of resorption | Medium |

Legend:
(−−) not detected, corresponds to the number of bacteria <10³
(+) weakly positive, corresponds to the number of bacteria 10³-10⁴
(++) mildly positive, corresponds to the number of bacteria 10⁴-10⁵
(+++) significantly positive, corresponds to the number of bacteria >10⁵

### Example 5. Permeation of CBD into Gingival Fibroblasts

Gingival fibroblasts (obtained from three healthy patients) were seeded in a 6-well plate (well area 9.6 cm²) and allowed to grow until the next day (confluence). CBD was applied to the cells at a concentration of 0.5 μM for 6 and 24 hours. At the end of the incubation period, 250 μl of medium was mixed with 250 μl of methanol with 1% (w/w) HCl and the cells were washed 2× with PBS (1 ml), scraped from 4 wells into PBS (phosphate buffer, 1 ml), centrifuged for 5 minutes at 3000 rpm. After centrifugation, PBS was removed and 0.5 ml of methanol with 1% (w/w) HCl was added and the cells were sonicated (cycle 0.5, amplitude 50, 10 times). The samples were then centrifuged for 2 minutes at 8000 rpm and the supernatant was pipetted into a clean tube and subjected to UHPLC/MS analysis.

UHPLC/MS analyses were performed on an ACQUITY I Class liquid chromatograph (Waters, Milford, MA, USA). It is a modular system consisting of a chromatographic pump (Binary Solvent Manager), autosampler (Sample Manager) and column thermostat (Column Manager). Kinetex Polar C18 (100×2.1 mm i.d., 2.6 μm; Phenomenex, CA, USA) was used as the analytical column. The system was operated with a gradient elution of the mobile phase (MFA 0.1% formic acid in water, MFB 0.1% formic acid in acetonitrile) at a flow rate of 0.6 ml/min and a temperature of 25° C. The gradient elution had the following parameters: 0 to 11 min 50 to 70% MFB, 11 to 12.5 min 70 to 100% MFB, 12.5 to 13 min 100 to 50% MFB, 13 to 16 min 50% MFB. The injection volume was 2 μl.

A high resolution Synapt G2-S mass spectrometer (Waters Corp., Manchester, UK) connected to the UPLC system via an electrospray ionization (ESI) interface was used for the metabolic study. The ion source was operated in positive ionization mode with a capillary voltage of 3.0 kV and a sample cone of 30 V. The source temperature and desolvation gas temperature were set at 120° C. and 320° C. The desolvation gas flow rate was 900 l/h. The data acquisition range was 50 to 1200 Da with a scan time of 0.2 s in MS^{E} mode (scanning function allowing simultaneous acquisition of low collision energy (2 eV) and high collision energy (15-30 eV) mass spectra in one experiment). The instrument was calibrated with sodium formate adducts in acetonitrile corrected for accurate mass measurement using an external standard (leucine-encephaline solution, 20 μg/l in water: acetonitrile: formic acid mixture (100:100:0.2, v/v/v), flow rate 5 μl/min). MassLynx V4.1 (Waters) was used as control software for data collection and evaluation, and subsequent data processing was performed using MetaboLynx XS Application Manager (Waters).

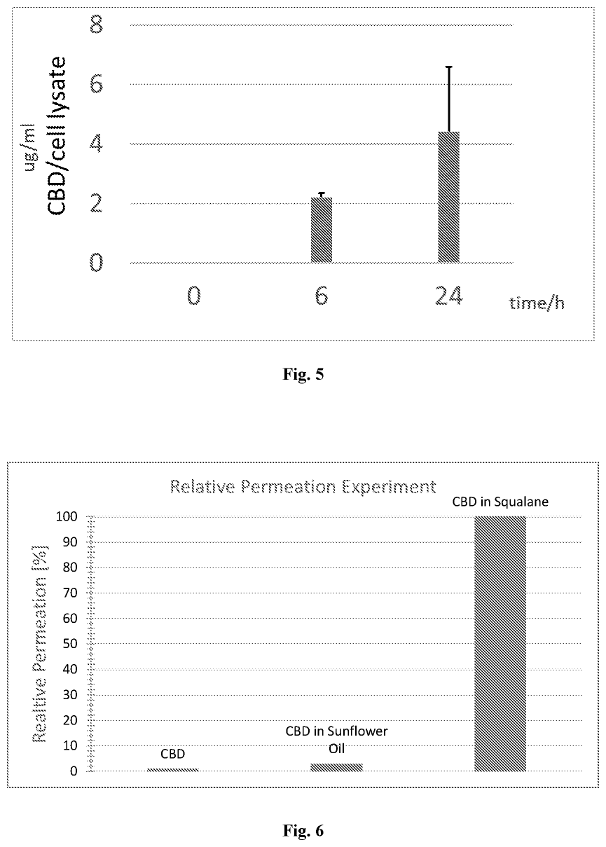

The results show that CBD is absorbed relatively quickly by gingival fibroblasts, which is also confirmed by the fact that CBD was not detected in the culture medium, but only in the examined gingival fibroblasts (FIG. 5). After 24 h of cultivation, no metabolites of CBD were identified, consistent with the fact that the gingiva is not a tissue with biotransformation potential. The fact that CBD is not metabolized by gingival fibroblasts also contributes to prolonging its anti-inflammatory effect.

### Example 6. Permeation of Dental Gels Containing CBD on a Synthetic Skin Model Strat-M®

The study was performed with dental gel samples having the following composition.

Dental gel with the active substance CBD and with squalane:

| component | weight % |
|---|---|
| Carbopol Ultrez 10 NF polymer | 0.50 |
| Glycerine | 10.00 |
| Squalane | 4.00 |
| Cannabidiol | 1.00 |
| Sodium methylparaben | 0.15 |
| Purified water | 84.35 |

### Procedure for Preparation of the Dental Gel:

1. Carbopol Ultrez 10 NF polymer is hydrated in water to form a whitish suspension.

2. Glycerine and sodium methylparaben are added to the suspension from step 1. The mixture is stirred.

3. pH of the mixture obtained in step 2. is adjusted by triethylamine to the value pH=6.0. Transparent gel is formed.

4. CBD is dissolved in squalane under heating to max. 60° C.

5. The squalane solution of CBD is mixed with the gel obtained in step 3., and the mixture is stirred.

6. To achieve a perfect dispersion of CBD in the gel, the mixture is homogenized at min. 3000 RPM for approx. 15 seconds.

Dental Gel with the Active Substance CBD and with Sunflower Oil:

| Component | weight % |
|---|---|
| Carbopol Ultrez 10 NF polymer | 0.50 |
| Glycerine | 10.00 |
| Sunflower oil | 4.00 |
| Cannabidiol | 1.00 |
| Sodium methylparaben | 0.15 |
| Purified water | 84.35 |

### Procedure for Preparation of the Dental Gel:

1. Carbopol Ultrez 10 NF polymer is hydrated in water to form a whitish suspension.

2. Glycerine and sodium methylparaben are added to the suspension from step 1. The mixture is stirred.

3. pH of the mixture obtained in step 2. is adjusted by triethylamine to the value pH=6.0. Transparent gel is formed.

4. CBD is dissolved in sunflower oil under heating to max. 60° C.

5. The sunflower oil solution of CBD is mixed with the gel obtained in step 3., and the mixture is stirred.

6. To achieve a perfect dispersion of CBD in the gel, the mixture is homogenized at min. 3000 RPM for approx. 15 seconds.

Dental Gel with the Active Substance CBD:

| Component | weight % |
|---|---|
| Carbopol Ultrez 10 NF polymer | 0.50 |
| Glycerine | 10.00 |
| Cannabidiol | 1.00 |
| Sodium methylparaben | 0.15 |
| Purified water | 88.35 |

### Procedure for Preparation of the Dental Gel:

1. Carbopol Ultrez 10 NF polymer is hydrated in water to form a whitish suspension.

2. Glycerine and sodium methylparaben are added to the suspension from step 1. The mixture is stirred.

3. pH of the mixture obtained in step 2. is adjusted by triethylamine to the value pH=6.0. Transparent gel is formed.

4. CBD powder is transferred into the gel obtained in step 3., and the mixture is stirred.

5. To achieve a perfect dispersion of CBD in the gel, the mixture is homogenized at min. 3000 RPM for approx. 15 seconds.

The experiments were performed in vertical Franz cells with a volume of 50 ml and a diffusion area of 2.5 cm². 5 g of sample was applied to the upper surface. The experiments were performed in triplicate at 37° C. and 400 rpm for 24 h. The receptor medium was phosphate buffer pH 7.4 with 5% BSA (bovine serum albumin) mimicking physiological conditions. The Strat-M® transdermal diffusion test model [T. Uchida, W. Kadhum, S. Kanai, T. Oshizaka, H. Todo, K. Sugibayashi Prediction of skin permeation by chemical compounds using the artificial membrane Strat-M®. Eur. J. Pharm. Sci. 2015; 67:113-118.] was used for the experiments. After 24 h, 1 ml sample was taken from the receptor space and analyzed by UHPLC/MS under the conditions specified in Example 5.

The results show that CBD diffuses best through a synthetic skin model from a formulation containing squalane as a solvent. Sunflower oil as a solubilizing medium was more than 90% less effective similarly as formulation without lipophilic component (FIG. 6).

### Industrial Applicability

The product according to the invention can be used in various forms as an effective means of oral hygiene combined with regular dental care providing protection against acute gingival inflammation caused by increased biofilm formation.

1. A therapeutic product comprising:
an active ingredient consisting of cannabidiol, and
squalane as solvent,
wherein the product is in the form of dental gel.

2. The product according to claim 1, wherein the weight ratio of squalane to cannabidiol is within the range of 2:1 to 20:1.

3. The product according to claim 1, comprising 0.1 to 5 weight % of cannabidiol.

4. The product according to claim 1, which further comprises hyaluronic acid or a pharmaceutically acceptable salt thereof.

5. The product according to claim 4, wherein cannabidiol and hyaluronic acid or a pharmaceutically acceptable salt thereof are contained in weight ratio 20:1 to 1:1.

6. The product according to claim 4, wherein hyaluronic acid or a pharmaceutically acceptable salt thereof has low to medium molecular weight within the range of 200 to 1800 kDa.

7. The product according to claim 1, which further comprises at least one auxiliary substance selected from gelling agents, solvents, water, stabilizers and flavorings.

8. The product according to claim 4, which comprises 0.1 to 5 weight % of cannabidiol, 0.01 to 2 weight % of hyaluronic acid or a pharmaceutically acceptable salt thereof, 1 to 10 weight % of squalane, 5 to 40 weight % of at least one gelling agent, and water q.s. 100 weight %.

9. The product according to claim 4, which comprises 0.5 to 2 weight % of cannabidiol, 0.05 to 1 weight % of hyaluronic acid or a pharmaceutically acceptable salt thereof, 3 to 8 weight % of squalane, 5 to 20 weight % of at least one gelling agent, and water q.s. 100 weight %.

10. The method of administering the product according to claim 1 in the treatment and/or prevention of gingival inflammation, periodontitis and periimplantitis.

11. The product according to claim 1, comprising 0.5 to 2 weight % cannabidiol.

12. The product according to claim 3, comprising up to 0.3 weight % of chlorhexidine gluconate or chlorhexidine digluconate.

13. The product according to claim 4, wherein cannabidiol and hyaluronic acid or a pharmaceutically acceptable salt thereof are contained in weight ratio 20:1 to 5:1.

14. The product according to claim 4, wherein cannabidiol and hyaluronic acid or a pharmaceutically acceptable salt thereof are contained in weight ratio 15:1 to 8:1.

15. The product according to claim 8, which comprises up to 0.3 weight % of chlorhexidine gluconate or chlorhexidine digluconate

16. The product according to claim 9, which comprises up to 0.3 weight % of chlorhexidine gluconate or chlorhexidine digluconate.
